(19) United States
(12) Patent Application Publication
(10) Pub. No.: US 2016/0243263 A1
(43) Pub. Date: Aug. 25, 2016

(54) PEGYLATED NANOPARTICLE COMPOSITIONS

(71) Applicant: BOARD OFf REGENTS, THE UNIVERSITY OF TEXAS SYSTEM, Austin, TX (US)

(72) Inventors: Jie ZHENG, Austin, TX (US); Jinbin LIU, Plano, TX (US)

(21) Appl. No.: 15/026,027

(22) PCT Filed: Sep. 16, 2014
(86) PCT No.: PCT/US14/55801

§ 371 Date: Mar. 30, 2016

Related U.S. Application Data
(60) Provisional application No. 61/884,514, filed on Sep. 30, 2013.

(51) Int. Cl. A61K 49/00

(52) U.S. Cl. CPC A61K 49/0093; A61K 49/0019; A61K 49/0054; A61K 49/0065

(57) ABSTRACT

An embodiment of the invention is directed to a composition comprising a noble metal nanoparticle, wherein the surface of the noble metal nanoparticle is coated with polyethylene glycol (PEG) having a size of 0.35 kDa to 5 kDa, and wherein the noble metal nanoparticle is about 2 nm to 10 nm in hydrodynamic diameter. In an embodiment of the invention, a therapeutic agent is also present on or in said nanoparticle.

This application claims benefit of priority to U.S. Provisional Application Ser. No. 61/884,514, filed Sep. 30, 2013, the entire contents of which are hereby incorporated by reference.

### STATEMENT REGARDING FEDERALLY-SPONSORED RESEARCH OR DEVELOPMENT

This invention was made with government support under Grant Nos. R21EB009853 and R21EB011762 awarded by the National Institutes of Health. The government has certain rights in the invention.

### BACKGROUND OF THE INVENTION

### I. Related Art

PEGylation is the most common and successful surface-chemistry strategy for reducing nonspecific accumulation, prolonging blood circulation and enhancing tumor targeting of inorganic nanoparticles (NPs) (Wang et al., 2010). These strengths fundamentally arise from the fact that the poly(ethylene glycol) (PEG) moiety on the particle surface creates steric hindrance for the serum protein (opsonin) adsorption and slows down the NP uptake by the mononuclear phagocytic system (MPS) in the reticuloendothelial system (RES) (Alexis et al., 2008). Consequently, retention time of the NPs in the blood is greatly prolonged, which enables the NPs to more effectively target tumors through well-known “enhanced permeability and retention (EPR)” effect (Matsumura and Maeda, 1986). However, the majority of PEGylated NPs still end up in RES organs after the circulation (Schipper et al., 2009), resulting in low targeting specificity (defined as the amount of NPs in tumor versus that in liver) (Aillon, et al., 2009). For example, gold nanocages (AuNCs) coated with 5 kDa PEG exhibited a tumor accumulation of 15.3±2.9% ID/g at 24 h post injection (p.i.), but nearly 65% ID/g and ˜30% ID/g of the AuNCs were found in the liver and spleen (Wang et al., 2013), respectively. Such long-term severe accumulation in RES potentially induces health hazards, hampering their clinical translation. Therefore, developing renal clearable PEGylated inorganic NPs that can be rapidly eliminated from the urinary system like their counterparts, clinically used small molecular contrast agents (Lorusso et al., 2001), is highly desired for their future applications in the clinical practices.

High RES uptake of PEGylated inorganic NPs essentially results from a significant increase in hydrodynamic diameters (HDs) due to PEG conjugation, preventing them from passing through the kidney filtration barrier with a size threshold of ˜5.5 nm (Longmire et al., 2008). For instance, for AuNPs with 2 nm diameter core, their HDs dramatically increased to 9˜10 nm after PEGylation, leading to 78% ID/g of AuNPs accumulation in the liver and 15.2% ID/g in the spleen (Arvizo et al., 2011). To develop renal clearable PEGylated inorganic NPs, Choi and coworkers investigated the influences of PEG lengths (DHLA-PEG, n=2, 3, 4, 8, 14, 22) on the renal clearance of QDs and observed efficient renal clearance from the QDs conjugated with PEG-4 (Choi et al., 2009). Other longer or shorter PEG ligands were not suitable to enhance renal clearance of QDs either because of their large HDs or due to low physiological stability (Choi et al., 2009). However, tumor targeting of the renal clearable PEGylated QDs is still not clear. Silica NPs coated with 0.5 kDa PEG were also renal clearable (Burns et al., 2009), but the passive tumor targeting efficiency was only 0.9% ID/g at 4 h p.i. (Benezra et al., 2011).

An alternative surface-chemistry strategy for making inorganic NPs renal clearable is to coat them with zwitterionic ligands. Choi and coworkers used cysteine (Choi et al., 2007) to functionalize 3 nm CdSe/ZnS QDs (cysteine-QDs), and the inventor's group used glutathione to stabilize 2.5 nm luminescent AuNPs (GS-AuNPs) (Zhou et al., 2010). Different from PEG ligands, these two zwitterionic ligands do not increase NP HDs significantly in physiological environments. After coating of cysteine or glutathione, the HDs of 3 nm QDs and 2.5 nm AuNPs were 4.9 nm and 3.4 nm, respectively, (Choi et al., 2007 and Liu et al., 2013) still smaller than kidney filtration threshold (KFT, ˜5.5 nm). Thus, they can be effectively eliminated from the urinary system (cysteine-QDs: >65% ID, 4 h p.i.; GS-AuNPs: >60% ID, 48 h p.i.) (Zhou et al., 2011), resulting in high targeting specificity. However, short blood retention time and low concentration of NPs in the blood pool decrease their effectiveness in passive tumor targeting through EPR effect. The tumor contrast was only enhanced ˜80% compared to normal tissues in the passive tumor targeting of renal clearable cysteine-QDs (Choi et al., 2010). Overall tumor targeting efficiency of GS-AuNPs was only 2.3% ID/g at 12 h p.i. (Liu et al., 2013). Low efficiencies in passive tumor targeting of renal clearable NPs with zwitterionic ligands have set up a new challenge in the delivery of inorganic NPs into the clinical practices. In addition, fundamental understandings of pros and cons of the PEGylation and zwitterionization in renal clearance and tumor targeting of renal clearable NPs are still missing.

### SUMMARY OF THE INVENTION

An embodiment of the invention is directed to a composition comprising a labeled noble metal nanoparticle, wherein the surface of the noble metal nanoparticle is coated with polyethylene glycol. More particularly, there is provided a composition comprising a labeled noble metal nanoparticle, wherein the surface of the noble metal nanoparticle is coated with polyethylene glycol (PEG) having a size of 0.35 kDa to 5 kDa, and wherein the noble metal nanoparticle is about 2 nm to 10 nm in hydrodynamic diameter. The label may be a radionuclide, a luminescent compound, a fluorescent compound, or combinations thereof. The noble metal may be selected from the group consisting of gold, silver, copper, platinum, palladium, or combinations thereof. The PEG may be 0.35 kDa, 1.0 kDa, 2.5 kDa or 5.0 kDa. The core of the nanoparticle may be about 2-8 nm. The hydrodynamic diameter of the nanoparticle may be about 2-7 nm. The ratio of PEG to noble metal surface atoms may be below 1:1. The nanoparticle may be detectable by PET, SPECT, CT, MRI, optical imaging, bioluminescence imaging, or combinations thereof, such as by fluorescence imaging or near infrared imaging.

The composition may comprise therapeutic agent or targeting ligand is attached to the nanoparticle, such as a therapeutic agent selected from the group consisting of an antibiotic, an antimicrobial, an antiproliferative, an antineoplastic, an antioxidant, an endothelial cell growth factor, a thrombin inhibitor, an immunosuppressant, an anti-platelet aggregation agent, a collagen synthesis inhibitor, a therapeutic antibody, a nitric oxide donor, an antisense oligonucleotide, a wound healing agent, a therapeutic gene transfer construct, an extracellular matrix component, a vasodilator, a thrombolytic, an antimetabolite, a growth factor agonist, an antimitotic, a statin, a steroid, a steroidal or nonsteroidal anti-inflammatory agent, an angiotensin converting enzyme (ACE) inhibitor, a free radical scavenger, a PPAR-gamma agonist, a small interfering RNA (siRNA), a microRNA, and an anti-cancer chemotherapeutic agent.

The blood retention half-life of the nanoparticle after administration of the nanoparticle to a subject may range from about 15 minutes to about 500 hours, or from about 60 minutes hours to about 48 hours. The renal clearance of the nanoparticle after administration of the nanoparticle to a subject may be greater than 10% in about 12 hours, or greater than 25% in about 12 hours, or from 10-25% in about 12 hours, or from 10-50% in about 12 hours, or from 10-75% in about 12 hours.

The PEG may have at least one thiol or dithiol terminal group, a reactive terminal group selected from NH₂ and COOH, or both. The terminal group may befurther conjugated to drug, nucleic acid, protein, lipid or carbohydrate.

In another embodiment, there is provided a method for detecting a cell or cellular component comprising (a) contacting the cell with a composition according to claim 1; and (b) monitoring the binding of the nanoparticle to the cell or a cellular component by at least one imaging technique. The label may be a radionuclide, a luminescent compound, a fluorescent compound, or combinations thereof. The noble metal may be selected from the group consisting of gold, silver, copper, platinum, palladium, or combinations thereof. The PEG may be 0.35 kDa, 1.0 kDa, 2.5 kDa or 5.0 kDa. The core of the nanoparticle may be about 2-8 nm, or 2-3 nm. The hydrodynamic diameter of the nanoparticle may be about 2-7 nm, or 2-4 nm. The ratio of PEG to noble metal surface atoms may be below 1:1. The nanoparticle may be detectable by PET, SPECT, CT, MRI, optical imaging, bioluminescence imaging, or combinations thereof, such as by fluorescence imaging or near infrared imaging.

In particular, the size of the PEG utilized for the present disclosure will range between 0.35 kDa and 5.0 kDa, inclusive, including intermediate lengths such as 0.5 kDa, 0.75 kDa, 1.0 kDa, 1.5 kDa, 2.0 kDa, 2.5 kDa, 3.0 kDa, 3.5 kDa, 4.0 kDa, 4.5 kDa, and 5.0 kDa. Density of PEG on the particle surface. the HD layer of PEG-AuNPs is about 0.5, 0.6, 0.7, 0.8, 0.9, 1.0, 1.1, 1.2, 1.3, 1.4, 1.5 or 1.6 nm with a calculated Flory radius about 1.5, 1.6, 1.7, 1.8, 19, or 2.0 nm of PEG in “mushroom” conformation (coiled/fold conformation) on the AuNP surface, suggesting a relatively low-density structure of PEG on the particle surface rather than a high-density extended structure.

The composition may comprise therapeutic agent or targeting ligand is attached to the nanoparticle, such as a therapeutic agent selected from the group consisting of an antibiotic, an antimicrobial, an antiproliferative, an antineoplastic, an antioxidant, an endothelial cell growth factor, a thrombin inhibitor, an immunosuppressant, an anti-platelet aggregation agent, a collagen synthesis inhibitor, a therapeutic antibody, a nitric oxide donor, an antisense oligonucleotide, a wound healing agent, a therapeutic gene transfer construct, an extracellular matrix component, a vasodialator, a thrombolytic, an antimetabolite, a growth factor agonist, an antimitotic, a statin, a steroid, a steroidal or nonsteroidal anti-inflammatory agent, an angiotensin converting enzyme (ACE) inhibitor, a free radical scavenger, a PPAR-gamma agonist, a small interfering RNA (siRNA), a microRNA, and an anti-cancer chemotherapeutic agent.

The blood retention half-life of the nanoparticle after administration of the nanoparticle to a subject may range from about 15 minutes to about 500 hours, or from about 60 minutes hours to about 48 hours. The renal clearance of the nanoparticle after administration of the nanoparticle to a subject may be greater than 10% in about 12 hours, or greater than 25% in about 12 hours, or from 10-25% in about 12 hours, or from 10-50% in about 12 hours, or from 10-75% in about 12 hours. The cell may be a tumor cell and/or the cellular component is a tumor cell component.

The PEG may have at least one thiol or dithiol terminal group, a reactive terminal group selected from NH₂ and COOH, or both. The terminal group may be further conjugated to drug, nucleic acid, protein, lipid or carbohydrate.

In yet another embodiment, there is provided a method for targeting a tumor cell comprising contacting the tumor cell an effective amount of a composition comprising noble metal nanoparticle, wherein the surface of the noble metal nanoparticle is coated with polyethylene glycol (PEG) having a size of 0.35 kDa to 5 kDa, and wherein the noble metal nanoparticle is about 2 nm to 10 nm in hydrodynamic diameter, and wherein conjugated with at least one therapeutic agent. The tumor cell may be located in a living subject. The composition may be administered orally, intravenously, nasally, subcutaneously, intramuscularly or transdermally.

The label may be a radionuclide, a luminescent compound, a fluorescent compound, or combinations thereof. The noble metal may be selected from the group consisting of gold, silver, copper, platinum, palladium, or combinations thereof. The PEG may be 0.35 kDa, 1.0 kDa, 2.5 kDa or 5.0 kDa. The core of the nanoparticle may be about 2-8 nm or 2-3 nm. The hydrodynamic diameter of the nanoparticle may be about 2-7 nm or 2-4 nm. The ratio of PEG to noble metal surface atoms may be below 1:1. The nanoparticle may be detectable by PET, SPECT, CT, MRI, optical imaging, bioluminescence imaging, or combinations thereof, such as by fluorescence imaging or near infrared imaging.

The composition may comprise therapeutic agent or targeting ligand is attached to the nanoparticle, such as a therapeutic agent selected from the group consisting of an antibiotic, an antimicrobial, an antiproliferative, an antineoplastic, an antioxidant, an endothelial cell growth factor, a thrombin inhibitor, an immunosuppressant, an anti-platelet aggregation agent, a collagen synthesis inhibitor, a therapeutic antibody, a nitric oxide donor, an antisense oligonucleotide, a wound healing agent, a therapeutic gene transfer construct, an extracellular matrix component, a vasodilator, a thrombolytic, an antimetabolite, a growth factor agonist, an antimitotic, a statin, a steroid, a steroidal or nonsteroidal anti-inflammatory agent, an angiotensin converting enzyme (ACE) inhibitor, a free radical scavenger, a PPAR-gamma agonist, a small interfering RNA (siRNA), a microRNA, and an anti-cancer chemotherapeutic agent.

The blood residence half-life of the nanoparticle after administration of the nanoparticle to a subject may range from about 15 minutes to about 500 hours, or from about 60 minutes hours to about 48 hours. The renal clearance of the nanoparticle after administration of the nanoparticle to a subject may be greater than 10% in about 12 hours, or greater than 25% in about 12 hours, or from 10-25% in about 12 hours, or from 10-50% in about 12 hours, or from 10-75% in about 12 hours.

The PEG may have a thiol terminal group, a reactive terminal group selected from NH₂ and COOH, or both. The terminal group may be further conjugated to drug, nucleic acid, protein, lipid or carbohydrate.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D. Characterization of NIR-emitting PEGylated AuNPs (PEG-AuNPs). (FIG. 1A) Scheme of the particle synthesis. (FIG. 1B) Typical TEM image showing that the synthesized PEG-AuNPs were monodispersed and (FIG. 1C) the core size was 2.3±0.3 nm, and dynamic light scattering (DLS) indicating a hydrodynamic diameter (HD) of 5.5±0.4 nm in aqueous solution. (FIG. 1D) Absorption and luminescent spectra of PEG-AuNPs (maximum emission: 810 nm; excitation peaks: 350, 550 nm).

FIGS. 2A-D. Biodistribution analysis of the passive tumor targeting in MCF-7 tumor bearing mice. (FIG. 2A) The biodistributions of PEG-AuNPs at 1, 12, 24, and 48 h p.i., respectively. (FIG. 2B) The time-dependent ratios of PEG-AuNPs concentration in tumor to that in liver in 48 h p.i. Inset showing a comparison of two probes, PEG-AuNPs and GS-AuNPs in the ratios of tumor to liver at 1, 12, and 24 h p.i. (FIG. 2C) The time-dependent tumor-to blood ratios of PEG-AuNPs within 48 h p.i. Inset was a comparison tumor to blood ratios of two probes at 1, 12, and 24 h p.i. (FIG. 2D) Pharmacokinetics of these two probes after the intravenous (IV) injection in 24 h, respectively. The distribution half-lives (t½α) of PEG-AuNPs and GS-AuNPs were 56.1±9.7 and 5.4±1.2 min, respectively. The elimination half-lives (t½β) of PEG-AuNPs and GS-AuNPs were 9.2±3.9 and 8.5±2.1 h, respectively (Data presented as mean±SD, n=3).

FIGS. 3A-C. Passive tumor targeting kinetics of PEG-AuNPs in MCF-7 tumor bearing nude mice. (FIG. 3A) In vivo NIR fluorescence images of the nude mice IV injected with PEG-AuNPs at 5, 12, 18, 24, and 48 h p.i. The tumor area of the mouse was marked with an arrow. (FIG. 3B) Time-dependent contrast index (CI) of PEG-AuNPs and GS-AuNPs showing that CI of PEG-AuNPs reach the desired value for tumor imaging (CI=2.5) slowly than that of GS-AuNP. (FIG. 3C) Accumulation and retention kinetics of PEG-AuNPs in normal tissue and tumor, respectively.

FIGS. 4A-B. Renal clearance kinetics of PEG-AuNPs in nude mice. (FIG. 4A) In vivo NIR fluorescence images of the nude mice IV injected with PEG-AuNPs collected at 2, 3, and 4 h p.i. The bladder area was marked with a circle. (FIG. 4B) Renal clearance kinetics of PEG-AuNPs and GS-AuNPs showing that PEG-AuNPs reached the maximum bladder intensity much slowly than GS-AuNPs. Inset showed the PEG-AuNPs found in the urine collected from the mice IV injected with PEG-AuNPs at 12 and 24 h p.i., respectively. The urine was measured by ICP-MS from 3 mice collected a group.

FIG. 5. The time-dependent luminescence spectra of a solution containing 50 mL 2.4 mM poly(ethylene glycol) methyl ether thiol (PEG-SH, ˜1 kDa) and 150 μL 1 M HAuCl₄ during the thermal reaction process. A solution with the volume of ˜250 μL was taken at each time point from the reaction mixture, and centrifuged at 21000 g for 1 min to remove the large aggregation before the fluorescence intensity measurement. The results showed that the luminescence intensity reached the maximum at ˜30 min during the reaction.

FIG. 6. The lifetimes of PEGylated AuNPs. Excited at 410 nm; measured to be 0.48 μs (49.4%) and 4.3 μs (50.6%).

FIG. 7. Photophysical stability of PEGylated AuNPs investigated by continuous monitoring luminescence spectra of the particles in PBS solution supplemented with 10% (v/v) FBS at 37° C. for 48 h. The fluorescence intensity of PEG-AuNP in PBS containing 10% (v/v) FBS were initially decreased by ˜20% compared to those in PBS solution without the addition of FBS. Interestingly, the fluorescence intensity recovered to the original intensity during 24 h incubation with 10% FBS in PBS buffer at 37° C., little change was observed in fluorescence intensity of PEG-AuNP for more than 48 h, implying that PEG-AuNP exhibit high photophysical stability in physiological environment.

FIG. 8. The luminescence images of urine. Mice were IV-injected with PEGylated AuNPs (A) PBS buffer as a control (B) 5 h p.i. The strong fluorescence intensity of the urine collected from the mouse IV injected with PEG-AuNP indicated that PEG-AuNPs were renal clearable. Imaging parameters: Excitation: 470/10 nm; Emission: 830/20 nm; Exposure time: 30 s.

### DETAILED DESCRIPTION OF THE DISCLOSURE

Inorganic NPs hold great potential for revolutionizing current diagnostic techniques. However, potential risks of inorganic NPs on human health remain a big challenge and require further understanding. In order to minimize the toxicity induced by the accumulation of NPs in reticuloendothelial system (RES) organs, significant efforts have been devoted to developing renal clearable nanomaterials by manipulating their sizes, shapes and surface chemistries. For instance, pioneering work on the renal clearable quantum dots (QDs) showed that the zwitterionic cysteine coated QDs with a hydrodynamic diameter (HD) of 5.5 nm could be rapidly cleared out through the urinary system within 4 h and less than ˜5% of the QDs were found in the liver. The origin of such efficient renal clearance was due to the fact that zwitterionic ligands can behave like poly(ethylene glycol) (PEG) ligands to minimize serum protein adsorption while maintaining the small HD of the QDs. Although the emergence of the new renal clearable inorganic NPs potentially further advances the translation of inorganic NPs into clinical practices, the library of renal clearable NPs is still limited to very few relatively inert nanomaterials, such as core-shell QDs and silica.

To address these challenges, the inventor has created a renal clearable PEGylated near-IR emitting AuNP (MR-emitting PEG-AuNPs) with photophysical properties, core sizes and high physiological stability identical to his previously reported NIR-emitting GS-AuNPs (Liu et al., 2013). By conducting head-to-head comparison of these two NPs in renal clearance and tumor targeting, the inventor was able to unravel pro and cons of these two surface chemistries in tumor imaging. The results show that PEG-AuNPs exhibit efficient renal clearance and low RES accumulation comparable to GS-AuNPs: >50% ID were excreted in the urine and only <4% ID/g of particles were accumulated in the liver 24 h p.i. However, these two types of renal clearable luminescent AuNPs are significantly different in tumor targeting: (1) PEG-AuNPs targeted tumor with an efficiency of ˜8% ID/g at both 1 and 12 h p.i., which is ˜3 times higher than that of GS-AuNPs and comparable to the high tumor targeting efficiencies of reported non-renal clearable NPs (Chen et al., 2010, Wang et al., 2013 and Zhang et al., 2009); (2) PEG-AuNPs also exhibit higher targeting specificity (2.4, at 12 h p.i.) than GS-AuNP (1.6, at 12 h p.i.) and most known renal clearable or non-renal clearable NPs. (Wang et al., 2010, Zhang et al., 2009 and Perrault et al., 2009; and (3) accumulation of PEG-AuNPs in the tumor and their clearance from normal tissues were 7- and 5-times slower than those of GS-AuNPs, respectively. As a result, contrast index (CI) of PEG-AuNPs reached a threshold for substantial tumor detection (CI=2.5) less quickly than that of GS-AuNPs. The fundamental understandings of the strengths and limitations of these two surface chemistries in tumor targeting of renal clearable AuNPs provide a foundation for design of new generation of renal clearable inorganic NPs for future clinical practices.

### I. NANOPARTICLES

The disclosure provides nanoparticle compositions comprising a nanoparticle, methods for preparing the nanoparticle compositions and methods of using the nanoparticle compositions. The compositions of the disclosure comprise noble metal nanoparticles, which are capable of emitting in the near infrared region of the light spectrum. In an embodiment, the noble metal nanoparticle comprises between 2 and 1000 noble metal atoms. In particular embodiments, the noble metal is selected from the group consisting of gold, silver, and copper. The properties of the nanoparticles enable excretion through the kidneys, as well as selective uptake and retention in tumors compared with normal tissues. This, along with the lack of in vivo toxicity, has resulted in a composition that is promising for translation to the clinic.

In certain embodiments of the claimed disclosure, a portion of the noble metal is present as its radioactive isotope. In an embodiment of the disclosure, the radioactive isotope is present at a concentration of up to 2% w/w of the noble metal. Embodiments of the disclosure comprise the radioactive isotopes of noble metal. In certain embodiments the radioactive isotope is ¹⁹⁸Au. In other embodiments, the radioactive isotope is ⁶⁴Cu. The presence of the radioactive isotope in the metal nanoparticle aids in the rapid monitoring of the pharmacokinetics of the NIR emitting radioactive particles and also offers an opportunity for in vivo SPECT imaging by emitting gamma rays, as well as in vivo PET imaging through β+ decay (positron emission).

A. Polyethylene Glycol

In certain embodiments of the disclosure, the surface of the noble metal nanoparticle is modified with poly(ethylene glycol) (PEG). PEG is a polyether compound with many applications from industrial manufacturing to medicine. The structure of PEG is (note the repeated element in parentheses):

H—(O—CH₂—CH₂)ₙ—OH

PEG is also known as polyethylene oxide (PEO) or polyoxyethylene (POE), depending on its molecular weight.

PEG, PEO, or POE refers to an oligomer or polymer of ethylene oxide. The three names are chemically synonymous, but historically PEG has tended to refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. PEGs are prepared by polymerization of ethylene oxide and are commercially available over a wide range of molecular weights from 300 g/mol to 10,000,000 g/mol. While PEG and PEO with different molecular weights find use in different applications, and have different physical properties (e.g., viscosity) due to chain length effects, their chemical properties are nearly identical. Different forms of PEG are also available, depending on the initiator used for the polymerization process—the most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete. Very high purity PEG has recently been shown to be crystalline, allowing determination of a crystal structure by x-ray diffraction. Since purification and separation of pure oligomers is difficult, the price for this type of quality is often 10-1000 fold that of polydisperse PEG.

PEGs are also available with different geometries. Branched PEGs have three to ten PEG chains emanating from a central core group. Star PEGs have 10 to 100 PEG chains emanating from a central core group. Comb PEGs have multiple PEG chains normally grafted onto a polymer backbone.

The numbers that are often included in the names of PEGs indicate their average molecular weights (e.g., a PEG with n=9 would have an average molecular weight of approximately 400 daltons, and would be labeled PEG 400. Most PEGs include molecules with a distribution of molecular weights (i.e., they are polydisperse). The size distribution can be characterized statistically by its weight average molecular weight (Mw) and its number average molecular weight (Mn), the ratio of which is called the polydispersity index (Mw/Mn). MW and Mn can be measured by mass spectrometry.

PEGylation is the act of covalently coupling a PEG structure to another larger molecule, for example, a therapeutic protein, which is then referred to as a PEGylated protein. PEG is soluble in water, methanol, ethanol, acetonitrile, benzene, and dichloromethane, and is insoluble in diethyl ether and hexane. It is coupled to hydrophobic molecules to produce non-ionic surfactants. In addition, thiolated PEG ligands can be composed of different terminal groups such as NH2-, COOH, others, which can be used for further bioconjugation.

In particular, the size of the PEG utilized for the present disclosure will range between 0.35 kDa and 5.0 kDa, inclusive, including intermediate lengths such as 0.5 kDa, 0.75 kDa, 1.0 kDa, 1.5 kDa, 2.0 kDa, 2.5 kDa, 3.0 kDa, 3.5 kDa, 4.0 kDa, 4.5 kDa, and 5.0 kDa. Density of PEG on the particle surface. the HD layer of PEG-AuNPs (1.6 nm) is much thinner than those of PEGylated 2 nm AuNPs (3.5-4 nm) (Aillon, et al., 2009), and comparable to the calculated Flory radius (F, ˜1.9 nm) of PEG (MW, 1 kDa) in “mushroom” conformation (coiled/fold conformation) on the AuNP surface (Della Rocca et al., 2011), suggesting a relatively low-density structure of PEG on the particle surface rather than a high-density extended structure (Zhou et al., 2010).

B. Targeting Ligands

In certain embodiments, the nanoparticle contains a ligand capable of specifically binding to at least one cellular component. The cellular component may be associated with specific cell types or having elevated levels in specific cell types, such as cancer cells or cells specific to particular tissues and organs. Accordingly, the nanoparticle can target a specific cell type, and/or provides a targeted delivery for the treatment and diagnosis of a disease. The ligand permits the nanoparticular to be used to identify, detect, target, or monitor a physical state or condition, such as a disease state or condition by binding to a cognate molecule or structure. For example, a ligand may be used to detect the presence or absence of a particular receptor, expression level of a particular receptor, or metabolic levels of a particular receptor. The ligand can be, for example, a peptide, a protein, a protein fragment, a peptide hormone, a sugar (i.e., lectins), a biopolymer, a synthetic polymer, an antigen, an antibody, an antibody fragment (e.g., Fab, nanobodies), an aptamer, a virus or viral component, a receptor, a hapten, an enzyme, a hormone, a chemical compound, a pathogen, a microorganism or a component thereof, a toxin, a surface modifier, such as a surfactant to alter the surface properties or histocompatability of the nanoparticle or of an analyte when a nanoparticle associates therewith, and combinations thereof.

### II. IMAGING

The present disclosure further encompasses methods of using the nanoparticles in order to study a biological state. The disclosure provides for a method of monitoring a molecule of interest by contacting the noble metal nanoparticle with a sample containing the molecule of interest. In a particular embodiment, the molecule of interest is present in a biological sample.

Nanoparticle compositions of the present disclosure are capable of emitting in the near infra-red range of 500 nm to 1100 nm. Compositions of the disclosure are capable of being detected by Single-Photon Emission Computed Tomography (SPECT) and fluorescence imaging techniques. Therefore, the nanoprobes of the disclosure can serve as dual-modality imaging probes.

In an embodiment of the invention, the compositions of the disclosure are used to monitor the surface of cell membranes. In certain embodiments of the disclosure, the cell membranes that are targeted are tumor cell membranes.

After administration of the nanoparticle to a subject, the blood residence half-life of the nanoparticles may range from about 2 hours to about 25 hours, from about 3 hours to about 20 hours, from about 3 hours to about 15 hours, from about 4 hours to about 10 hours, or from about 5 hours to about 6 hours. Longer blood residence half-life means longer circulation, which allows more nanoparticles to accumulate at the target site in vivo. Blood residence half-time may be evaluated as follows. The nanoparticles are first administered to a subject (e.g., a mouse, a miniswine or a human). At various time points post-administration, blood samples are taken to measure nanoparticle concentrations through suitable methods.

An embodiment of the disclosure is directed to a metal nanoparticle that is renal clearable. In certain embodiments of the disclosure, the compositions demonstrate greater than 50% renal clearance within 48 hours of administration.

An embodiment of the disclosure is directed to a method for detecting a component of a cell comprising the steps of: contacting the cell with a composition comprising a labeled noble metal nanoparticle, wherein the surface of the noble metal nanoparticle is coated with PEG, and wherein the noble metal nanoparticle is about 2 nm to 10 nm in diameter, optionally wherein a portion of the noble metal is present as a radionuclide; and monitoring the binding of the nanoparticle to the cell or a cellular component by at least one imaging technique. The imaging techniques include PET, SPECT, CT, MRI, optical imaging, bioluminescence imaging.

### III. THERAPY

In an embodiment of the disclosure, a therapeutic agent is attached to the nanoparticle of the present disclosure. The therapeutic agent an agent capable of treating a disease state or disorder, and may be selected from the group consisting of antibiotics, antimicrobials, antiproliferatives, antineoplastics, antioxidants, endothelial cell growth factors, thrombin inhibitors, immunosuppressants, anti-platelet aggregation agents, collagen synthesis inhibitors, therapeutic antibodies, nitric oxide donors, antisense oligonucleotides, wound healing agents, therapeutic gene transfer constructs, extracellular matrix components, vasodialators, thrombolytics, antimetabolites, growth factor agonists, antimitotics, statins, steroids, steroidal and nonsteroidal anti-inflammatory agents, angiotensin converting enzyme (ACE) inhibitors, free radical scavengers, PPAR-gamma agonists, small interfering RNAs (siRNAs), microRNAs, and anti-cancer chemotherapeutic agents.

A further embodiment of the disclosure is directed to a method for targeting a tumor cell comprising administering to the subject an effective amount of a composition comprising a noble metal nanoparticle, wherein the surface of the noble metal nanoparticle is coated with PEG, and wherein the noble metal nanoparticle is about 2 nm to 10 nm in diameter, optionally wherein a portion of the noble metal is present as a radionuclide, and optionally wherein the nanoparticle further comprises a ligand is capable of binding a tumor marker, and wherein at least one therapeutic agent is conjugated to the nanoparticle.

### IV. NANOPARTICLES COMPOSITIONS

A. Preparing Nanoparticles

The present disclosure further encompasses methods for the preparation of the noble metal nanoparticle having the characteristics as described herein. In one embodiment, the method of preparing a noble metal nanoparticle comprises the steps of: a) combining an aqueous solution comprising a noble metal, and an aqueous solvent to create a combined solution; b) adding a first ligand; c) mixing the combined solution to allow the formation of a noble metal nanoparticle; and d) adjusting the pH of the combined solution using acid or base. In certain embodiments of the disclosure, the aqueous solution of the noble metal contains an amount of a radioactive isotope of the noble metal. In an embodiment of the disclosure, the radioactive isotope is present in the nanoparticle at a concentration of up to 2% w/w of the noble metal.

In certain embodiments of these methods, a reducing agent is added to the combined solution to reduce the noble metal nanoparticle. In particular, the reducing agent is selected from the group comprising a chemical reducing agent, light, or a combination thereof. In certain embodiments of these methods, light can be used as a reducing agent to photoreduce the noble metal nanoparticles. In certain other embodiments of these methods, a chemical reducing agent can be used as a reducing agent. In one embodiment, light is used in combination with a reducing agent to photoreduce the noble metal nanoparticles.

In particular, the aqueous solution comprising a noble metal ion used in the preparation of the compounds is selected from the group consisting of AgNO₃, HAuCl₄.nH₂O, and CuCl₂.nH₂O. In one embodiment, the aqueous solution comprising a noble metal is AgNO₃. In another embodiment, the aqueous solution comprising a noble metal is HAuCl₄.nH₂O. In a further embodiment, the aqueous solution comprising a noble metal is CuCl₂.nH₂O.

In one embodiment, the aqueous solution comprising a noble metal is HAuCl₄.nH₂O, a reducing agent is added to the combined solution along with a ligand, the pH adjusted, and the combined solution is mixed for at least one hour to allow the formation of the gold nanoparticle. In another embodiment, the pH adjusted, combined solution is mixed for about 48 hours or longer (up to several months) to allow the formation of a luminescent gold nanoparticle. In another embodiment, noble metal nanoparticles are created through photoreduction through irradiation with visible or ultraviolet light to allow the formation of a gold, silver or copper nanoparticle. In this work, the inventors used the weak reducing property of thiolated ligands (PEG-SH) to reduce gold ions to gold nanoparticles.

B. Pharmaceutical Compositions

In certain embodiment, the nanoparticles of the present disclosure will be formulated as pharmaceutical composition, i.e., suitable for administration to patients. Pharmaceutical compositions of the present disclosure comprise an effective amount of a nanoparticular dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The candidate substance may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, subcutaneously, subconjunctival, intravesicularlly, mucosally, intrapericardially, intraumbilically, intraocularally, orally, locally, via inhalation (e.g., aerosol inhalation), via injection, via infusion, via continuous infusion, via localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The candidate substance may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in particular embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the candidate substance is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### V. DEFINITIONS

As used herein, the term “noble metal” refers to the group of elements selected from the group consisting of gold, silver, and copper and the platinum group metals (PGM) platinum, palladium, osmium, iridium, ruthenium and rhodium. In certain particular embodiments of the present disclosure, the noble metal is selected from the group consisting of gold, silver, and copper. In other particular embodiments, the noble metal is silver. In other particular embodiments, the noble metal is gold. In other particular embodiments, the noble metal is copper.

As used herein, the term “nanoparticle” refers to an association of 2-1000 atoms of a metal. Nanoparticles may have diameters in the range of about 2 to about 5 nm. In other particular embodiments, the nanoparticles comprise approximately 2-1000, approximately 2-500, approximately 2-250, approximately 2-100, approximately 2-25 atoms, or approximately 2-10 atoms.

As used herein, the term “labeled” refers to an entity, e.g., a nanoparticle, that carries a molecule capable of detection, either directly or indirectly.

As used herein, the term “hydrodynamic diameter” refers to the diameter of the particles in the solution, which includes the actual size and hydrodynamic water layer.

As used herein, the term “nanoparticle core” refers to metal core of the particles.

As used herein, the term “about” refers to the stated value, plus or minus 5% of that stated value.

### VI. EXAMPLES

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Materials and Equipment.

Hydrogen Tetrachloroaurate used for the synthesis of NIR emitting PEGylated gold nanoparticles (PEG-AuNPs) was purchased from Fisher Scientific (U.S.). All the other chemicals were obtained from Sigma-Aldrich and used as received unless specified. The luminescence spectra were collected by a PTI QuantaMaster™ 30 Fluorescence Spectrophotometer (Birmingham, N.J.). Absorption spectra were acquired from a Varian 50 Bio UV-Vis spectrophotometer. Hydrodynamic diameter of the nanoparticles (NPs) in the aqueous solution was analyzed by a Brookhaven 90Plus Dynamic Light Scattering Particle Size Analyzer (DLS). Transmission electron microscopy (TEM) images were obtained with a 200 kV Jeol 2100 transmission electron microscope. The in vivo near-infrared fluorescence imaging was acquired from a Carestream Molecular imaging system In-Vivo FX PRO (U.S.).

Animals and Tumor Implantation.

The animal studies were performed according to the guidelines of the University of Texas System Institutional Animal Care and Use Committee. The female nude mice (Athymic NCr-nu/nu, strain code: 01B74) of 6-8 weeks old, weighing 20˜25 g, were purchased from the National Cancer Institute (NCI) Frederick National Laboratory. The animals were housed in ventilated cages under standard environmental conditions (23±1° C., 50±5% humidity and a 12/12 hr light/dark cycle) with free access to water and standard laboratory food.

The human breast cancer cell line MCF-7 was cultured in Minimum Essential Medium (MEM) with 10% (v/v) fetal bovine serum (FBS) and 1% (v/v) penicillin-streptomycin at 37° C. in humidified atmosphere containing 5% CO₂. The cell suspension (in MEM with 10% (v/v) FBS) was then mixed 3:1 (v/v) with Matrigel and injected subcutaneously upper near the mammary fat pad (MFP) area of the mouse with a volume of 100 μL dense suspension (containing about 1×10⁶ cells) for each mouse. The tumor was allowed to grow ˜2 weeks to reach a palpable size (˜6-8 mm) before the studies.

Synthesis of NIR Emitting PEGylated Gold Nanoparticles (PEG-AuNPs).

The NIR emitting PEGylated AuNPs were synthesized by a thermal reduction method. Briefly, 50 mL of 2.4 mM poly(ethylene glycol) methyl ether thiol (PEG-SH, ˜1 kDa) solution was mixed with 150 μL 1M HAuCl₄ under vigorous stirring. The mixture was then heated in a 95° C. oil bath for 30 min until the fluorescence intensity of the solution reached the maximum monitored by a fluorescence spectrometer. The resulting solution was cooled to room temperature and centrifuged at 21,000 g to remove the large aggregates after the reaction. The PEG-AuNPs were collected from the supernatant by freeze-drying for 24 hr, and resuspended in 300 μl PBS buffer, followed by purifying with NAP-5 column for several times in PBS buffer before the animal study.

Biodistribution and Pharmacokinetics Studies of PEGylated AuNPs.

To evaluate the tissue distributions of PEG-AuNP, the MCF-7 tumor bearing mice were IV injected with PEG-AuNP (7 mg/mL) and sacrificed at 1, 12, 24 and 48 hr p.i. (n=3 for each time point). The collected organs and tumors were weighted and the biodistributions of gold (% injection dose/g, % ID/g) were measured by the ICP-MS method described as follows. The weighted organs and tumors were completely lysed in 3 mL freshly made aqua regia in screw capped glass bottles (10 mL) separately for 24 h, followed by heating the samples at 115° C. in an oil bath until the complete evaporation of the aqua regia. The residue for each sample was then re-dissolved in 10 mL 0.05 M HCl, followed by sonicating for 30 min. The resulting samples were then further diluted with 0.05 M HCl and analyzed by a Perkin Elmer-SCIEX ELAN 6100 DRC Mass Spectrometry. As for the pharmacokinetics studies, the nude mice IV injected with 200 μL PEG-AuNP (7 mg/mL) were blood sampled from the retro-orbital sinus at 2, 5, 10, 30 min, 1, 3, 5, 8, 12, and 24 hr p.i. The gold concentrations in the blood samples injected with PEG-AuNP were measured using ICP-MS method, and the procedures were the same as described above.

In Vivo NIR Fluorescence Imaging of Tumor Targeting and Result Analysis.

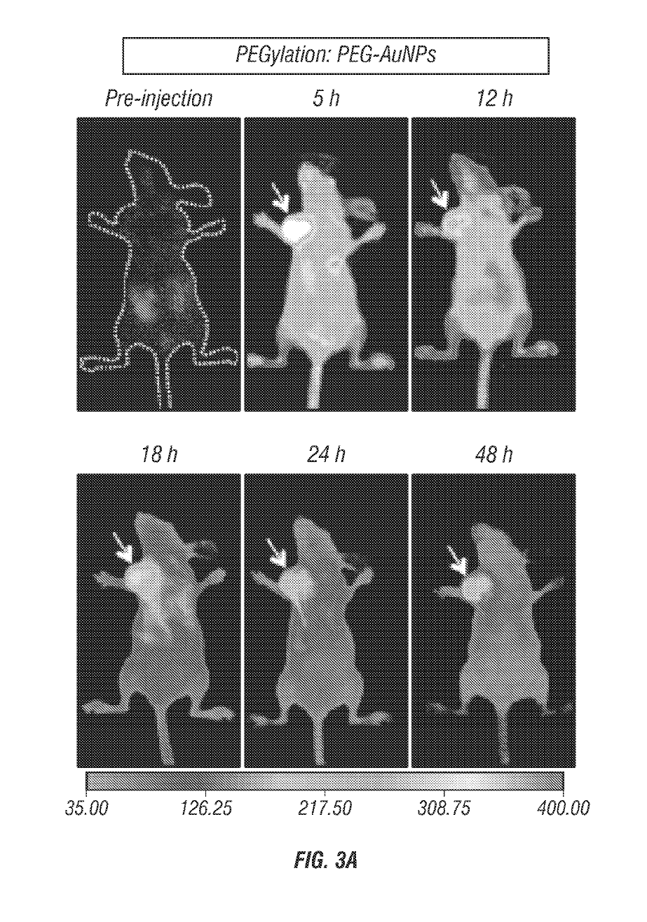

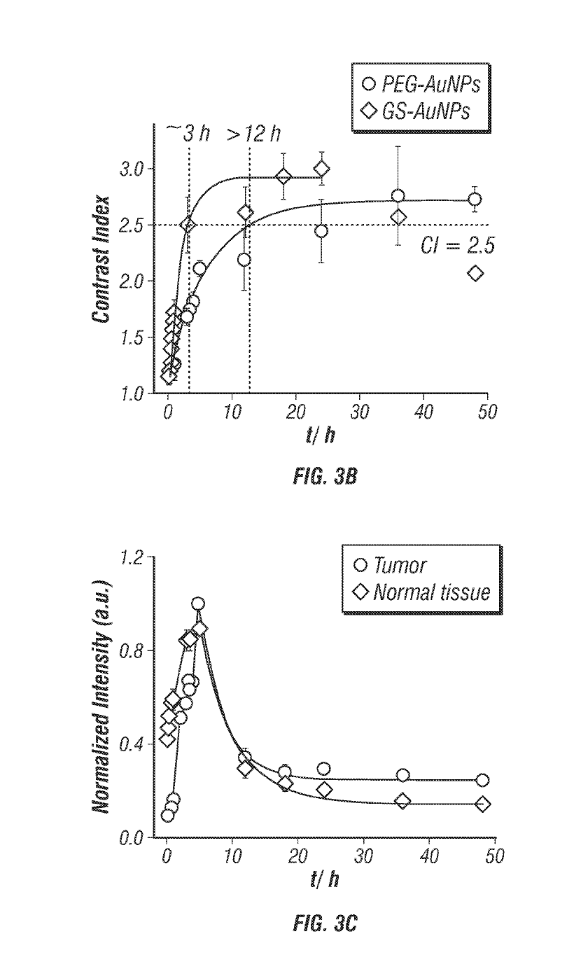

The MCF-7 tumor bearing nude mice were anesthetized using 1.5-3% isofluorane in oxygen flowing at 0.8 L/min˜5 min prior to IV injection. Each mouse was pre-imaged before the IV injection of PEG-AuNPs. After IV injection of 200 μl PEG-AuNP (20 mg/ml), the MCF-7 tumor bearing nude mouse was imaged at different p.i. time points (as described in the corresponding figures) in 48 hr using the exact same imaging parameters (Excitation: 470/10 nm; Emission: 830/20 nm; Exposure time: 30 s) at each time point. The representative images were shown in FIG. 3A.

To evaluate the accumulation and retention kinetics of the particles in tumors and normal tissues, the time dependent contrast index (CI), retention in tumors and normal tissues were defined the same as a previous report (Liu et al., 2013). Briefly, the CI value was measured according to the formula CI=(fluorescence intensity of tumor area−autofluorescence)(fluorescence intensity of normal contralateral region−autofluorescence) (Jiang et al., 2004). The pixels of the tumor area were corresponding to the tumor location identified from the white light image. The fluorescence of normal contralateral region was calculated by the mean intensity value of 3 selected areas with the same pixels of the tumor area on the contralateral region of the mouse. The autofluorescence from the corresponding region measured before the IV injection of PEG-AuNPs.

The tumor intensity was defined as (fluorescence intensity of tumor area—fluorescence intensity of normal contralateral region), and the normal tissue background intensity was defined as (fluorescence intensity of normal contralateral region—autofluorescence). The fluorescence intensity described in the kinetics study was normalized by setting the corresponding maximum fluorescence intensity of the time point to be 1. The results of time-dependent CI values, and retention kinetics of PEG-AuNP in tumor and normal tissues were shown in FIGS. 3B-C.

In Vivo Renal Clearance Kinetics Studies of PEGylated AuNPs.

After the IV injection of 200 μL NIR emitting PEG-AuNP (˜20 mg/mL), the nude mouse without tumor was imaged continuously for 5 h with the same imaging parameters (Excitation: 470/10 nm;

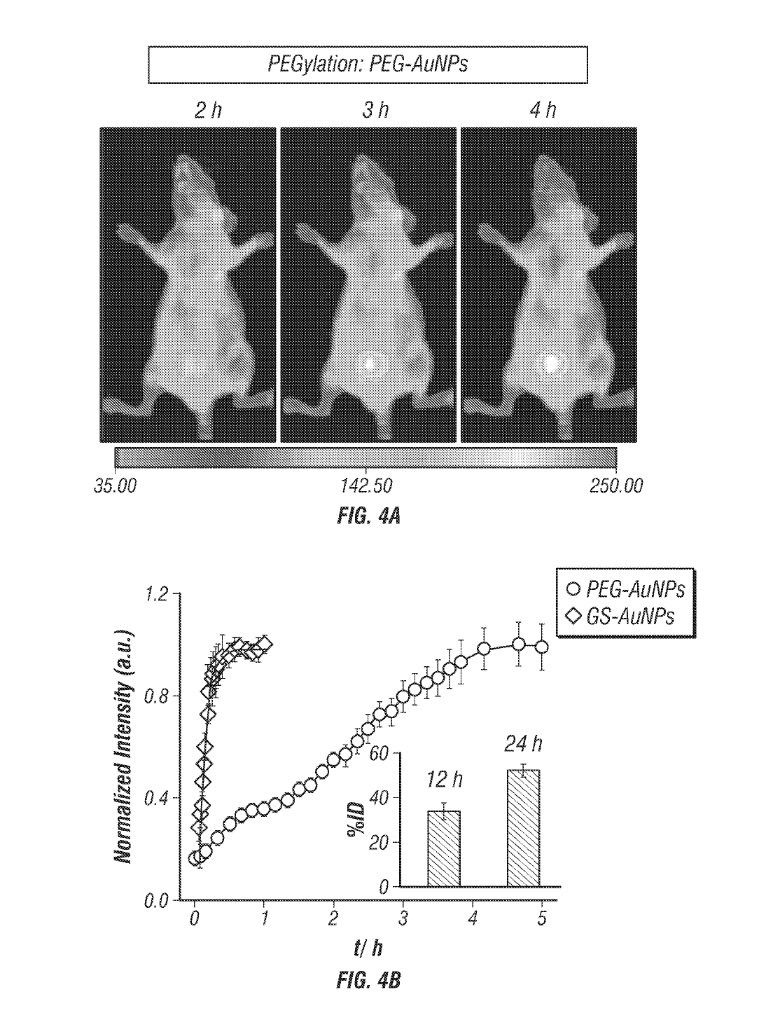

Emission: 830/20 nm; Exposure time: 30 s). The mouse was anesthetized using 1.0˜1.5% isofluorane in oxygen flowing at 0.8 L/min during the imaging process. The representative images were shown in FIG. 4A. The renal clearance kinetics was obtained by measuring the mean intensities of bladder at different p.i. time points using the regions of interest (RIO) analysis with a Carestream molecular imaging software (Version 5.0.7.22).

### Example 2

### Results

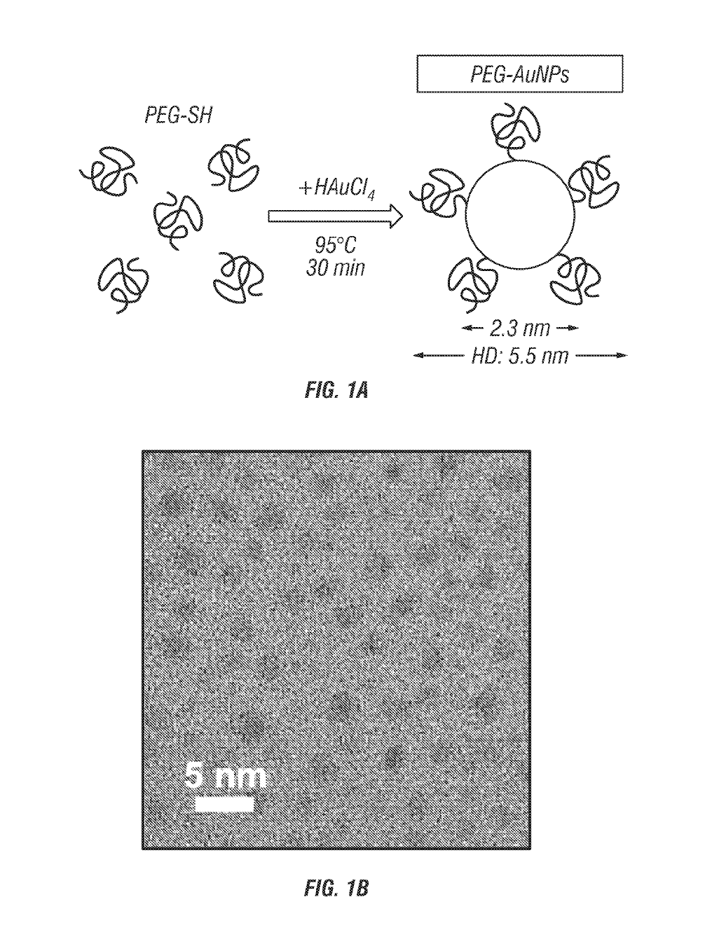

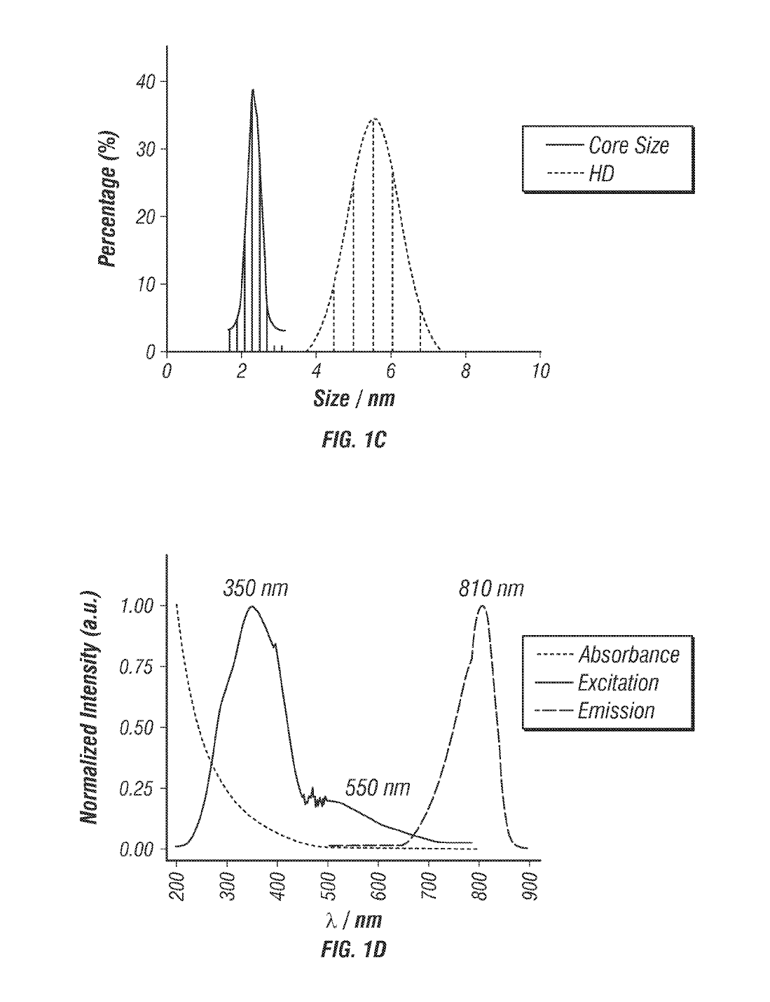

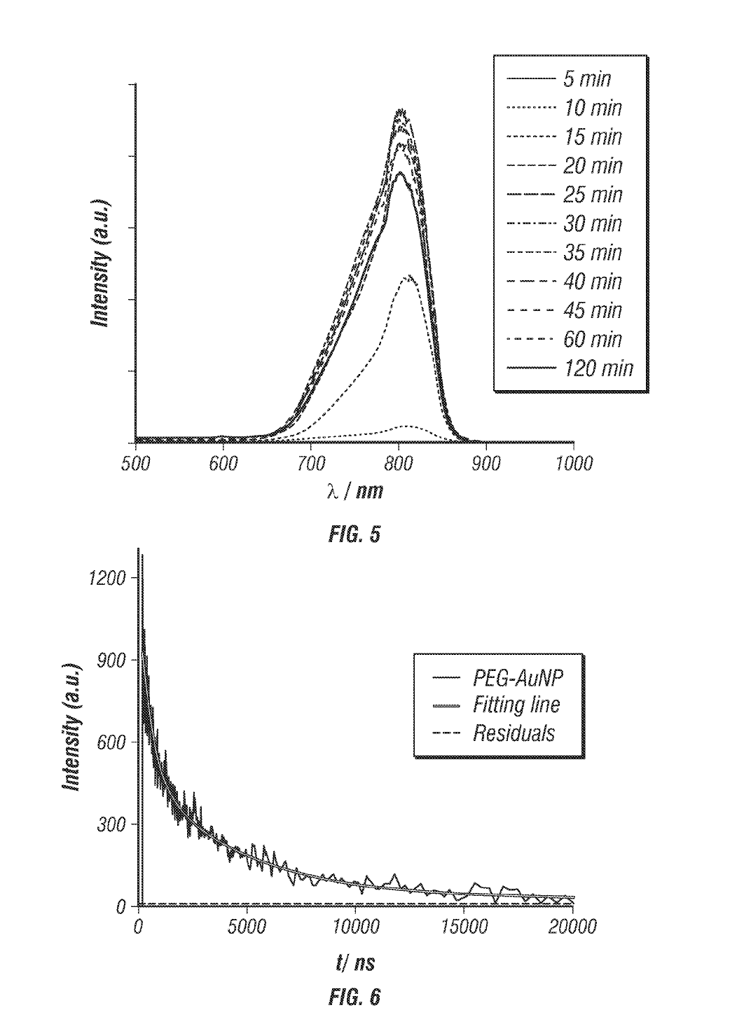

NIR-emitting PEG-AuNPs were synthesized by thermally reducing HAuCl₄ in the presence of thiolated PEG (PEG-SH) ligands with molecular weight (MW) of 1 kDa (˜22 units) in aqueous solution (FIG. 1A, and supporting information). Briefly, the mixture of PEG-SH and HAuCl₄ at a 1:1.25 ratio was heated in oil bath at 95° C. under vigorously stirring. The reaction was stopped until the fluorescence intensity of the solution reached the maximum (˜30 min) (FIG. 5). The particles were concentrated first and then purified with NAP-5 column to remove the free PEG ligands and large aggregates. The core size of PEG-AuNPs was measured to be 2.3±0.3 nm, almost exactly the same as that of GS-AuNPs (2.5 nm), but their HD (5.5±0.4 nm) was slightly larger than the HD of GS-AuNPs (3.4 nm) (FIGS. 1B-C). Such a larger increase in HD after PEGylation in comparison with zwitterionic ligand coating was in agreement with previous findings (Wang et al., 2010). The PEG coating layer of PEG-AuNPs (1.6 nm) is thinner than those of PEGylated 2 nm AuNPs (Arvizo et al., 2011) and 3.2 nm QDs (Choi et al., 2009), and comparable to the calculated Flory radius (F, ˜1.9 nm) of PEG (MW, 1 kDa) in “mushroom” conformation on the AuNP surface, (Jokerst et al., 2011) suggesting a relatively low density structure of PEG on the particle surface rather than a high density extended structure (Wang et al., 2010).

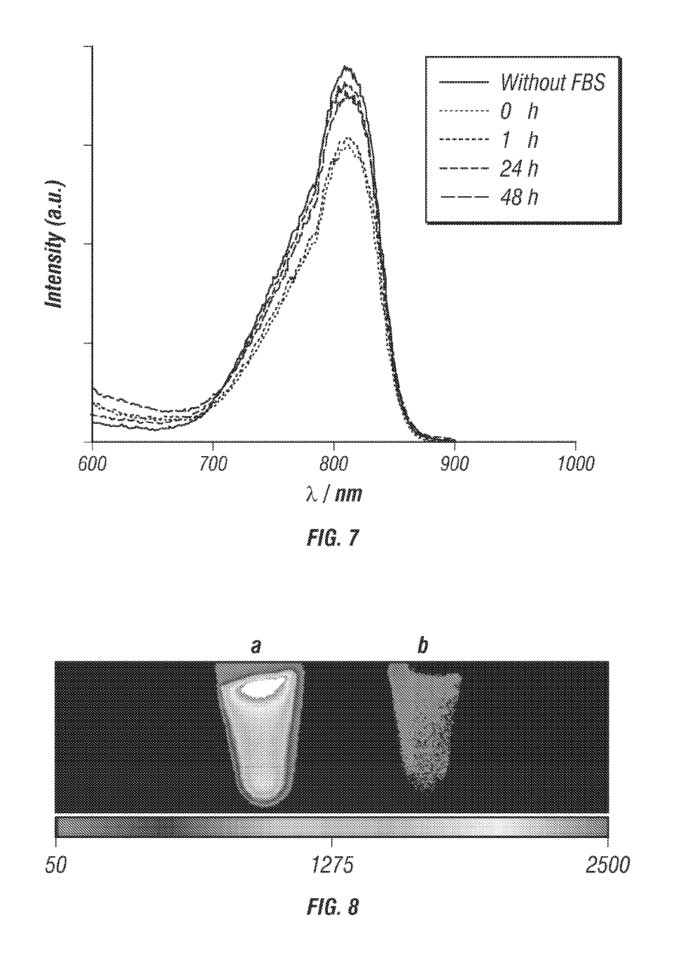

The PEG-AuNPs exhibited strong emission with a maximum at 810 nm and broad excitation with two peaks at 350 and 550 nm (FIG. 1D), respectively. The large Stokes shifts (>460 nm) and the microsecond emission lifetimes (0.48 μs (49.4%) and 4.3 μs (50.6%), FIG. 6) indicated that the NIR emission was generated from the quantized transitions between surface states formed via hybridization of p orbitals of sulfur atoms and the sp band of surface Au (I) atoms (Zhou et al., 2010; Zhu et al., 2008). These NIR emission and broad excitation of PEG-AuNPs, avoiding interference of autofluorescence background and blood absorption of animals, offer an exceptional opportunity for investigating their in vivo behaviors with NIR fluorescence imaging. In addition, the core size, photophysical properties and high physiological stability (FIG. 7) of NIR-emitting PEG-AuNPs are almost identical to those of NIR-emitting GS-AuNPs, making it possible to head-to-head compare their tumor targeting and renal clearance under the exactly same imaging conditions; so that potential different effects of these two well-known surface passivation strategies, PEGylation and zwitterionization, on passive tumor targeting of renal clearable NPs can be unraveled.

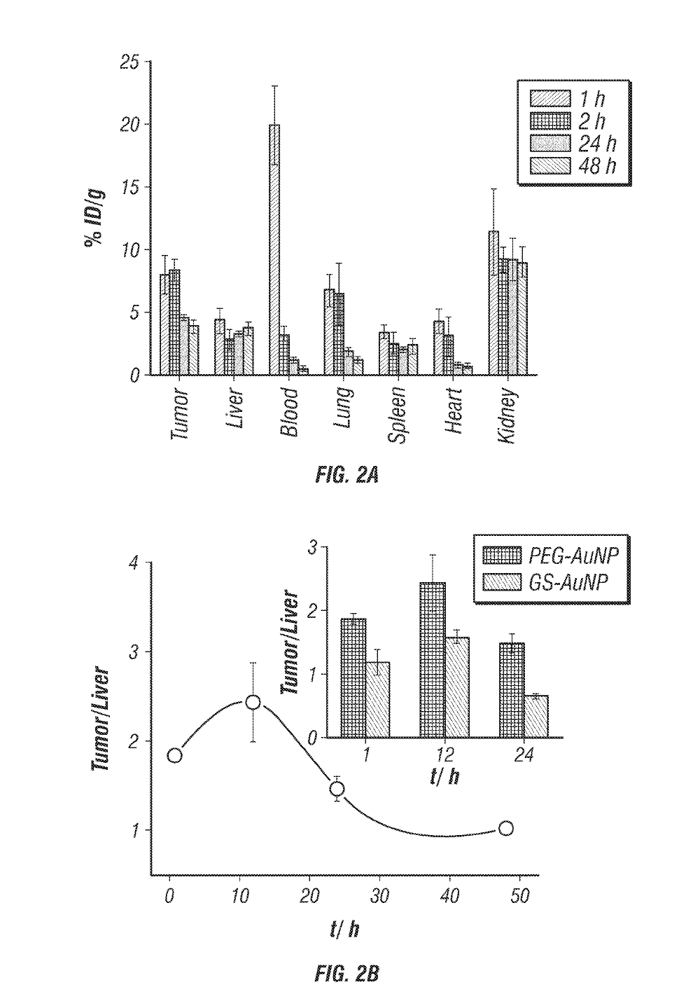

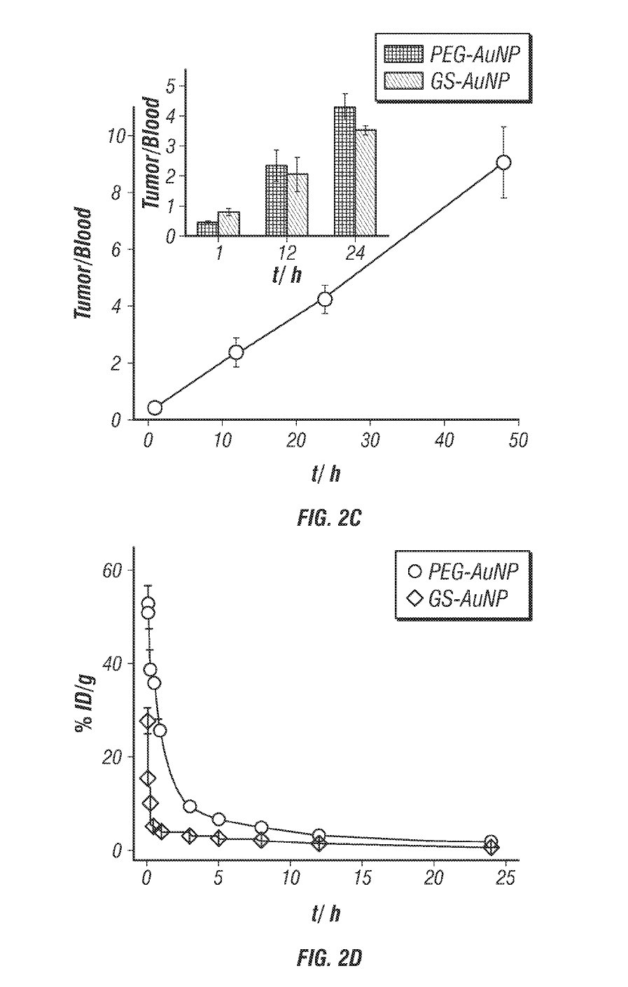

To quantify the tumor targeting efficiency and specificity, biodistribution of PEG-AuNPs at the selected p.i. time points (1, 12, 24, and 48 hr p.i.) was analyzed by ICP-mass spectrometry (FIG. 2A). The accumulations of PEG-AuNPs in tumor were determined to be 8.0±1.5% and 8.3±0.9% ID/g at 1 and 12 hr p.i., respectively, which were ˜3 times higher than that of GS-AuNPs at the same p.i. time points, and were also significantly higher than those of previously reported renal clearable silica NPs (0.9% ID/g) (Benezra et al., 2011). It should be noted that the targeting efficiency of ˜8% ID/g was even higher or comparable to those of reported non-renal clearable NPs, such as PEGylated AuCNs used as photothermal transducers for cancer treatment (5.7% ID/g), (Chen et al., 2010) PEGylated 20 nm AuNPs (6.63% ID/g) (Zhang et al., 2009), and six-armed PEGylated Ag2S QDs (10% ID/g) (Hong et al., 2012). In terms of liver uptake, the maximum accumulation was 4.35% ID/g at 1 hr p.i. and the amount remained rough constant (3.27˜3.76% ID/g) during subsequent 48 h, which is comparable to the liver accumulation of GS-AuNPs. Since PEG-AuNPs in the tumor were cleared faster than those in the liver, the targeting specificity were time-dependent (FIG. 2B) and reached its maximum of 2.4±0.4 at 12 hr p.i., higher than that of GS-AuNPs (1.6±0.1) (Liu et al., 2013) and nearly two orders better than most known non-renal clearable NPs (Wang et al., 2010, Zhang et al., 2009; Perrault et al., 2009). The difference in tumor and liver uptake behaviors between PEG-AuNPs and GS-AuNPs, suggested that PEGylation of renal clearable NPs prefers enhancing tumor targeting rather than increasing non-specific accumulation.

The origin of high tumor targeting efficiency of these NIR-emitting PEG-AuNPs is attributed to their strong EPR effect enhanced by conjugation of PEG ligands. To determine how well EPR effect functions, the tumor/blood ratio of probes is a typical parameter to be measured (Matsumura and Maeda, 1986). Shown in FIG. 2C, the tumor to blood ratio of PEG-AuNPs monotonously increased and reached to 9.0±1.3 at 48 hr p.i., which is comparable to the data of many known non-renal clearable NPs with strong EPR effect. For example, the tumor to blood ratio (4.2±0.5) of the NPs at 24 hr was even higher than that of reported PEG coated 33 nm AuNCs (3.81±1.08) at the exactly same time point (Wang et al., 2013). The tumor to blood ratios of PEG-AuNPs were also higher than those of GS-AuNPs at 12 and 24 hr p.i. (FIG. 2C), respectively, suggesting that PEGylation can enhance EPR effect more than that of zwitterionization. While EPR effect is often considered as a unique strength of non-renal clearable NPs because they can escape the kidney filtration and retain in the blood at a high concentration (Iyer et al., 2006; Modi et al., 2006), the observed high tumor/blood ratio of PEG-AuNPs clearly indicated that strong EPR effect can also be achieved by renal clearable inorganic NPs through PEGylation.

The enhanced EPR effect of PEG-AuNPs is fundamentally due to prolonged blood retention of the NPs. A classical two-compartment pharmacokinetics was observed for PEG-AuNPs, which were 56.1±9.7 min, and 9.2±3.9 hr for distribution half-life (t½α) and elimination half-life (t½β), respectively (FIG. 2D). The t½α of PEG-AuNP was one order longer than that of GS-AuNP, and the t½β of PEG-AuNP and GS-AuNP were comparable. In addition, area under the cure (AUC) of PEG-AuNP (142.8% ID·h/g) was 3 times larger than that of GS-AuNP (47.2% ID·h/g). Such high concentration of PEG-AuNPs in the blood is fundamentally responsible for their high tumor targeting efficiency.

Subsequently, passive tumor targeting kinetics of NIR-emitting PEG-AuNPs was measured through real-time imaging of the particles accumulation in the tumor. As shown in FIG. 3A, the mouse became visible due to the intravenous (IV) injection of the PEG-AuNPs. However, it was difficult to identify the tumor at the initial imaging stage because of the strong fluorescence background from the normal tissues. With time increasing, more NPs were accumulated in the tumor and normal tissues, and the preference of accumulation of NPs in tumor to the normal tissues eventually led to the clear discrimination of tumor at 12 hr p.i. The fluorescence background was significantly reduced due to the continuous clearance of non-targeted NPs from the normal tissues at 18 hr p.i., making the tumor much more evident. The tumor remained detectable at 48 hr p.i, indicating that the PEGylated AuNPs did retain in the tumor for a very long time.

The time-dependent accumulation and clearance of PEG-AuNPs in tumor and normal tissue govern the quality of tumor detection. Contrast index (CI) is a general parameter used in tumor imaging to evaluate how well the tumor could be distinguished from normal tissues due to the probes (Jiang et al., 2004). As shown in FIG. 3B, it took >12 hr for PEG-AuNPs to reach the CI threshold value (CI=2.5) for tumor detection, (Andreev et al., 2007) which is much longer than that of GS-AuNPs (˜3 hr) according to a previous study (Liu et al., 2013). The reason why the GS-AuNPs rapidly reached the imaging CI threshold value was because of the short retention time of the GS-AuNPs in normal tissue even though their tumor targeting efficiency was much lower than that of PEG-AuNPs.

To gain more quantitative understanding of tumor targeting of PEG-AuNPs, the inventor quantified accumulation and clearance kinetics of PEG-AuNPs and compared the kinetics with NIR emitting GS-AuNPs. As shown in FIG. 3C, fluorescence intensities of normal tissues reached its maximum at 5 hr p.i., while our previous studies show that it only took <10 min for GS-AuNPs to reach its maximum (Liu et al., 2013). The clearance kinetics of PEGylated AuNPs in the normal tissues exhibited a monoexponential decay with a half-life of 4.1±0.2 hr, more than 5 time longer than that of GS-AuNPs (43.4±6.6 min) (FIG. 3C). Subsequent analysis of tumor retention kinetics showed that PEG-AuNPs reached their maximum accumulation at the tumor site at 5 hr p.i., more than 7 times more slowly than that of GS-AuNPs (˜40 min), (Liu et al., 2013) which is consistent with their slow diffusion in the normal tissues. Once the emission intensity of the tumor reached its maximum, PEG-AuNPs, like many other NPs (Perrault et al., 2009) or nanosized proteins, (Matsumura and Maeda, 1986) slowly released back to the blood stream.

Tumor clearance kinetics of PEG-AuNPs can be fitted with monoexponential decay with half-life of 3.7±0.8 hr. Interestingly, unlike GS-AuNPs, more than 76% of which remain in the tumor at 24 hr p.i., only ˜30% of the PEG-AuNPs were retained in the tumor. The slow clearance of PEG-AuNPs from the normal tissues was also consistent with the observation in their renal clearance kinetics. While GS-AuNPs were rapidly excreted into the bladder to make bladder intensity reach the maximum within 1 hr p.i., very little accumulation of PEG-AuNPs in the bladder was observed in the same time window. The bladder area started to become visible at 3 hr p.i., and reached the maximum intensity at 5 hr p.i. (FIG. 4). The urine collected from the mouse injected with PEG-AuNPs within the first 5 hr p.i. showed a strong NIR emission, further confirming that the particles were renal clearable (FIG. 8). Although the PEG-AuNPs showed a slow renal clearance in the initial stage, it should be noted that a comparable amount of PEG-AuNPs and GS-AuNPs were found in the urine 24 hr p.i., suggesting that the slow clearance of PEG-AuNPs from the normal tissues results from their slow diffusion in the body rather than delayed uptake by RES organs.

In summary, the inventor synthesized a renal clearable NIR-emitting PEGylated AuNP with photophysical properties and core size identical to zwitterionic GS-AuNP. Systematic studies on renal clearance, pharmacokinetics and passive tumor targeting of PEG-AuNPs show that PEG based passivation chemistry has significant impacts on in vivo behaviors of renal clearable NIR-emitting AuNPs, distinct to the glutathione based zwitterionic one. PEGylation can significantly enhance tumor targeting efficiency and specificity of AuNPs without sacrificing the low accumulation in RES organs due to their prolonged retention time and high concentration in the blood. The obtained renal clearable PEG-AuNPs successfully addressed a long-term challenge of current renal clearable NP, making inorganic NPs more effectively and specifically target the tumor without loss of their renal clearance. However, the limitation of PEGylation in tumor imaging is that it took much longer time for PEG-AuNPs to reach desired CI threshold value than that of GS-AuNPs because of their slow tumor accumulation and normal-tissue clearance. The fundamental understandings of differences of these two distinct surface chemistries provide a foundation for developing a new generation of renal clearable inorganic NPs that can be eventually translated into the clinical practices with minimized nanotoxicity.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VII. REFERENCES

The following references, to the extent that they provide exemplary procedural or other details supplementary to those set forth herein, are specifically incorporated herein by reference:
Aillon et al., Adv. Drug Deliv. Rev., 61, 457-466, 2009.
Alexis et al., Mol. Pharmaceut., 5, 505-515, 2008.
Andreev et al., Proc. Natl. Acad. Sci. USA, 104, 7893-7898, 2007.
Arvizo et al., PLoS One, 6, e24374, 2011.
Benezra et al., J. Clin. Invest., 121, 2768-2780, 2011.
Burns et al., Nano Lett., 9, 442-448, 2009.
Choi et al., Nano Lett., 9, 2354-2359, 2009.
Choi et al., Nat. Biotechnol., 25, 1165-1170, 2007.
Choi et al., Nat. Nanotechnol., 5, 42-47, 2010.
Della Rocca et al., Angew. Chem. Int. Edit., 50, 10330-10334, 2011.
Hong et al., Angew. Chem. Int. Edit., 51, 9818-9821, 2012.
Iyer et al., Drug Discov. Today, 11, 812-818, 2006.
Jiang et al., Proc. Natl. Acad. Sci. USA, 101, 17867-17872, 2004.
Jokerst et al., Nanomedicine-Uk, 6, 715-728, 2011.
Liu et al., J. Am. Chem. Soc., 135, 4978-4981, 2013.
Longmire et al., Nanomedicine-Uk, 3, 703-717, 2008.
Lorusso et al., Invest. Radiol., 36, 309-316, 2001
Matsumura and Maeda, Cancer Res., 46, 6387-6392, 1986.
Modi et al., Curr. Pharm. Des., 12, 4785-4796, 2006.
Perrault et al., Nano Lett., 9, 1909-1915, 2009.
Schipper et al., Small, 5, 126-134, 2009.
Wang et al. Pharmacol. Res., 62, 90-99, 2010.
Wang et al., Acs Nano, 6, 5880-5888, 2012.
Wang et al., Nano Lett., 13, 581-585, 2013.
Zhang et al., Biomaterials, 30, 1928-1936, 2009.
Zhou et al., Angew. Chem. Int. Edit., 50, 3168-3172, 2011.
Zhou et al., J. Phys. Chem. C, 114, 7727-7732, 2010.
Zhu et al., J. Am. Chem. Soc., 130, 5883-5885, 2008.

1. A composition comprising a labeled noble metal nanoparticle, wherein the surface of the noble metal nanoparticle is coated with polyethylene glycol (PEG) having a size of 0.35 kDa to 5 kDa, and wherein the noble metal nanoparticle is about 2 nm to 10 nm in hydrodynamic diameter.

2. The composition of claim 1, wherein the label is a radionuclide, a luminescent compound, a fluorescent compound, or combinations thereof.

3. The composition of claim 1, wherein the noble metal is selected from the group consisting of gold, silver, copper, platinum, palladium, or combinations thereof.

4. The composition of claim 1, wherein the PEG is 0.35 kDa, 1.0 kDa, 2.5 kDa or 5.0 kDa.

5. The composition of claim 1, wherein the core of the nanoparticle is about 2-8 nm, and/or the hydrodynamic diameter of the nanoparticle is about 2-7 nm.

6. The composition of claim 1, wherein the ratio of PEG to noble metal surface atoms is below 1:1.

7. The composition of claim 1, wherein the nanoparticle is detectable by PET, SPECT, CT, MM, optical imaging, bioluminescence imaging, or combinations thereof.

8. The composition of claim 7, wherein the optical imaging is fluorescence imaging.

9. The composition of claim 7, wherein the optical imaging is near infrared imaging.

10. The composition of claim 1, wherein said PEG has at least one thiol terminal group and/or one terminal group selected from NH₂ and COOH.

11. The composition of claim 1, wherein a therapeutic agent or targeting ligand is attached to the nanoparticle.

12. The composition of claim 11, wherein the therapeutic agent is selected from the group consisting of an antibiotic, an antimicrobial, an antiproliferative, an antineoplastic, an antioxidant, an endothelial cell growth factor, a thrombin inhibitor, an immunosuppressant, an anti-platelet aggregation agent, a collagen synthesis inhibitor, a therapeutic antibody, a nitric oxide donor, an antisense oligonucleotide, a wound healing agent, a therapeutic gene transfer construct, an extracellular matrix component, a vasodilator, a thrombolytic, an antimetabolite, a growth factor agonist, an antimitotic, a statin, a steroid, a steroidal or nonsteroidal anti-inflammatory agent, an angiotensin converting enzyme (ACE) inhibitor, a free radical scavenger, a PPAR-gamma agonist, a small interfering RNA (siRNA), a microRNA, and an anti-cancer chemotherapeutic agent.

13. The composition of claim 1, wherein blood retention half-life of the nanoparticle after administration of the nanoparticle to a subject ranges from about 15 minutes to about 500 hours.

14. The composition of claim 13, wherein the blood retention half-life of the nanoparticle ranges from about 60 minutes hours to about 48 hours.

15. The composition of claim 1, wherein renal clearance of the nanoparticle after administration of the nanoparticle to a subject is greater than 10% in about 12 hours.

16. The composition of claim 15, wherein renal clearance of the nanoparticle after administration of the nanoparticle to a subject is greater than 25% in about 12 hours.

17. A method for detecting a cell or cellular component comprising:
(a) contacting the cell with a composition according to claim 1; and
(b) monitoring the binding of the nanoparticle to the cell or a cellular component by at least one imaging technique.

18. The method of claim 17, wherein the label is a radionuclide, a luminescent compound, a fluorescent compound, or combinations thereof.

19. The method of claim 17, wherein the noble metal is selected from the group consisting of gold, silver, copper, platinum, palladium, or combinations thereof.

20-30. (canceled)

31. A method for targeting a tumor cell comprising contacting the tumor cell an effective amount of a composition comprising noble metal nanoparticle, wherein the surface of the noble metal nanoparticle is coated with polyethylene glycol (PEG) having a size of 0.35 kDa to 5 kDa, and wherein the noble metal nanoparticle is about 2 nm to 10 nm in hydrodynamic diameter, and wherein conjugated with at least one therapeutic agent.

32-45. (canceled)
